(19) United States
(12) Patent Application Publication
(10) Pub. No.: US 2026/0334767 A1
(43) Pub. Date: Oct. 29, 2026

(54) MECHANISMS AND METHODS FOR CONTROL OF ACTUATION MOTION LIMIT OF A CONTINUUM ROBOT

(71) Applicant: CANON U.S.A., INC., MELVILLE, NY (US)

(72) Inventors: Daisuke Yamada, Cambridge, MA (US); Zachary Hamilton Haubert, Somerville, MA (US); Brian Ninni, Woburn, MA (US)

(21) Appl. No.: 19/189,771

(22) Filed: Apr. 25, 2025

(51) Int. Cl. A61B 1/005; A61B 1/00; A61B 1/008; A61M 25/01

(52) U.S. Cl. CPC A61B 1/0055; A61B 1/00006; A61B 1/00057; A61B 1/0053; A61B 1/008; A61M 25/0116

(57) ABSTRACT

Disclosed are an apparatus, an actuator, a surgical apparatus and methods for controlling same, with the actuator including a track, a tractor that moves along the track, a motor, first and second limit sensors, and first and second hard stops. Operation of the motor moves the tractor along the track between the first and second hard stops, with the first and second limit sensors spaced apart therebetween.

### FIELD

The present disclosure relates generally to medical devices and, more particularly, to devices and methods for image orientation during navigation of a continuum robot to guide interventional tools and instruments, such as endoscopes and catheters.

### BACKGROUND

Endoscopy is a medical procedure allowing remote inspection, manipulation, and/or treatment of internal organs using flexile devices inserted through natural orifices. The complexity of anatomical pathways in a patient, the limited flexibility of existing instruments, and the need to use multiple instruments may limit such procedures. Endoscopes are generally composed of a passive proximal section and an active distal section. The proximal passive section can be rigid, semi-rigid, or flexible. The active distal section includes a steerable tip that is remotely actuated by control wires connected to actuation wheels located on the handle of the device. Endoscopes may be equipped with an imaging device, e.g., a chip on tip (COT) camera, a light source, irrigation and/or suction channels, and at least one instrument channel for passing interventional tools. Typical endoscopic instruments have at least three degrees of freedom (3DOF) which allow for insertion, rotation and grasping operations. However, confined workspaces, limited visibility, and varied orientation of target organs limit usability of these systems and require one or more highly skilled endoscopists to perform dexterous tasks. For example, four hands may be required to manually control the endoscope and its instruments during certain procedures. To that end, an endoscopist may need to master a combination of accurate tip angulations, shaft management, and instrument insertion procedures, while communicating with an endoscopist assistant to actuate the instrument and hold the endoscopic shaft in a correct position. Techniques have been proposed and robotic endoscope systems have been developed to alleviate the complexity of such procedures.

In minimally invasive medical procedures, certain techniques make use of flexible tubular instruments, e.g., a catheter, inserted through natural orifices or surgical incisions to deliver imaging tools, e.g., an endoscope, diagnostic tools, e.g., forceps, brush, and/or needle, or therapeutic tools, e.g., an ablation probe, to reach a target tissue location. The catheter may contain drive wires to control its shape and tip orientation in a continuous curving structure. When mechanically advanced and actuated, the catheter may constitute a continuum robot.

To assist a clinician in navigating to the target through natural passageways, e.g., airways, or through surgically created passageways, anatomical models are prepared using pre-operative or intra-operative imaging, e.g., computed tomography (CT) and/or magnetic resonance imaging (MRI). Thus, target tissue and a planned path may be identified and navigation can be virtually simulated. Use of three-dimensional (3D) tracking technologies, e.g., electromagnetic and/or shape sensing, with sensors embedded in the catheter may allow for real-time localization in the registered anatomical model during a procedure.

Navigation of the catheter may be guided by a camera or videoscope arranged at the distal tip of the catheter shaft. A display device, such as a liquid crystal display (LDC) monitor provided in a console or attached to a wall, may display an image of the camera's field of view (FOV) to assist the user in navigating the catheter through a patient's anatomy to reach the target site. The orientation of the camera view, the controller, and the catheter may be mapped (calibrated) before inserting the catheter into the patient's body. As the physician or other user manipulates the endoscope within the anatomy to obtain a real view, the camera transfers the camera's FOV image to the display device. Ideally, the displayed image should allow the physician to relate to the endoscopic image as if the physician's own eyes were actually inside the endoscope cavity.

WO 2023/154825 A 1 to Haubert discusses a magnetic breakaway for a medical device, with a breakaway connection including an actuator portion on the actuator and a medical device portion on the medical device. The actuator portion may include an actuator support, an actuator side ferromagnetic block, and a slider element; and the medical device portion may include a medical device support, a medical device side ferromagnetic block, and a slider element. One of the actuator side ferromagnetic block and the medical device side ferromagnetic block may include a magnet, the actuator is coupled to the medical device by a magnetic coupling force, and the actuator may decouple from the medical device when a decoupling force is greater than a predetermined amount.

US 2018/0303319 to Ikeda et al. discusses a flexible tube insertion apparatus that includes an insertion section, a stiffness variable unit that changes a bending stiffness of the insertion section and a detection unit that detects an advance that is a movement toward a distal end of the insertion section and a retreat that is a movement toward a proximal end of the insertion section. A controller controls the stiffness variable unit to change the bending stiffness of the insertion section to a first bending stiffness when the detection unit has detected the advance of the insertion section, and controls the stiffness variable unit to change the bending stiffness of the insertion section to a second bending stiffness higher than the first bending when the detection unit has detected the retreat of the insertion section.

Conventional systems and methods describe the travel limits of actuation motion, but do not provide mechanisms and methods to restrict and correct the actuation motion.

### SUMMARY

Thus, the present disclosure overcomes shortcomings of conventional systems by providing a surgical apparatus with mechanisms and methods for restricting and correcting actuation motion.

An aspect of the present disclosure provides an actuator that includes a track, a tractor, a motor, an encoder, at least one limit sensor, and a controller in operational communication with the motor, the encoder, and the at least one limit sensor. Operation of the motor moves the tractor along the track at least between a first position, a second position and a third position. The first position is set on a first side of the least one limit sensor. The second position spaced apart from the first position. The third position is set on a second side of the least one limit sensor that is opposite the first side. The encoder is configured to output data indicating a position of the tractor at at least at one of the first position, the second position and the third position.

Another aspect of the present disclosure provides a method for controlling an actuator that includes a track, a tractor, a motor, an encoder, and at least one limit sensor, with the method including operating the motor to move the tractor along the track at least between a first position, a second position and a third position; and outputting, by the encoder, data indicating a position of the tractor at at least at one of the first position, the second position and the third position, with the first position being set on a first side of the least one limit sensor, the second position being spaced apart from the first position, and the third position being set on a second side of the least one limit sensor that is opposite the first side.

A further aspect of the present disclosure provides an actuator that includes a plurality of tracks; a plurality of physical stops at one end of each track of the plurality of tracks; a plurality of tractors, with each tractor of the plurality of tractors configured to travel along a respective track of the plurality of tracks; a plurality of motors, with each motor of the plurality of motors configured to drive a respective tractor; a plurality of encoders, with each encoder of the plurality of encoders being configured to encode a position of a respective tractor along the respective track; a plurality of limit sensors; and a controller in operational communication with the plurality of motors, the plurality of encoders, and the plurality of limit sensors.

Operation of a first motor moves a first tractor along a first track at least between a first position, a second position and a third position, provided in such order along the first track. A first encoder of the plurality of encoders is configured to output data indicating a position of the first tractor at least between the first position and the second position. The first position is set as a software limit. The second position is set at a first predetermined position at or adjacent to a first limit sensor along the first track. The third position is set at a second predetermined position at or adjacent to a first physical stop along the track. A range of travel of the first tractor is based on the set software limit. In response to the first tractor being within the range of travel, the controller is configured to permit driving of the motor. In response to the first tractor being outside of the range of travel, the controller is configured to control output of an error state notification. In response to detecting the first tractor at the second position, the controller restricts the first motor from moving the first tractor towards the third position.

These and other embodiments, objects, features, and advantages of the present disclosure will become apparent upon reading the following detailed description of exemplary embodiments of the present disclosure, when taken in conjunction with the appended drawings, and provided claims.

### DESCRIPTION OF THE DRAWINGS

The accompanying drawings, which are incorporated herein and form part of the specification, illustrate various embodiments, objects, features, and advantages of the present disclosure.

FIG. 1 illustrates a medical environment for use of a robotic catheter system.

FIG. 2 is a block diagram of the robotic catheter.

FIG. 3A is an exemplary embodiment of a steerable catheter.

FIG. 3B and FIG. 3C illustrate catheter tip manipulations by actuating one or more bending segments of the steerable catheter.

FIG. 4 is a block diagram of the robotic catheter system.

FIG. 5 is a block diagram of components of the system controller and/or display controller, according to an embodiment.

FIG. 6 illustrates movement of a tractor along a track, according to an embodiment.

FIG. 7A illustrates preclusion of movement of the tractor to one end of the predetermined range of travel, according to an embodiment.

FIG. 7B illustrates preclusion of movement of the tractor to another end of the predetermined range of travel, according to an embodiment.

FIG. 8A illustrates preclusion of movement of the tractor outside the predetermined range of travel, according to an embodiment.

FIG. 8B illustrates preclusion of movement of the tractor to outside another end of the predetermined range of travel, according to an embodiment.

FIG. 9A illustrates preclusion of movement of the tractor beyond a hard stop outside of the predetermined range of travel, according to an embodiment.

FIG. 9B illustrates preclusion of movement of the tractor beyond another hard stop outside of the predetermined range of travel, according to an embodiment.

FIG. 10 illustrates a tractor with a force sensor, according to an embodiment.

FIG. 11 is a block diagram of a motor drive process according to an embodiment.

FIG. 12 is a block diagram of operation of a redundant limit mechanism according to an embodiment.

FIG. 13 illustrates components for maintaining the tractor within the predetermined range of travel, according to an embodiment.

FIG. 14A is a plan view illustrating components of a system according to an embodiment.

FIG. 14B is a front perspective view illustrating components of a system according to an embodiment.

FIG. 14C is a rear perspective view illustrating components of a system according to an embodiment.

Throughout the figures, similar reference numerals and characters, unless otherwise stated, are used to denote like features, elements, components, or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The present disclosure has several embodiments and relies on patents, patent applications and other references for details known to those of the art. Therefore, when a patent, patent application, or other reference is cited or repeated herein, it should be understood that the patent, patent application, or other reference is incorporated by reference in its entirety for all purposes as well as for the proposition that is recited.

The present disclosure generally relates to medical devices, and it exemplifies embodiments of an endoscope or catheter, and more particular to a steerable catheter controlled by a medical continuum robot (M CR). The embodiments of the endoscope or catheter and portions thereof are described in terms of their state in a three-dimensional space. As used herein, the term position refers to a location of an object or a portion of an object in a three-dimensional space (e.g., three degrees of translational freedom along Cartesian X, Y, Z coordinates); the term orientation refers to the rotational placement of an object or a portion of an object (three degrees of rotational freedom—e.g., roll, pitch, and yaw); the term posture refers to the position of an object or a portion of an object in at least one degree of translational freedom and to the orientation of that object or portion of object in at least one degree of rotational freedom (up to six total degrees of freedom); the term shape refers to a set of posture, positions, and/or orientations measured along an elongated body of the MCR.

As known in the field of medical devices, the terms proximal and distal are used with reference to the manipulation of an end of an instrument extending from the user to a surgical or diagnostic site. In this regard, the term proximal refers to the portion (e.g., a handle) of the instrument closer to the user, and the term distal refers to the portion (tip) of the instrument further away from the user and closer to a surgical or diagnostic site. It will be further appreciated that, for convenience and clarity, spatial terms such as vertical, horizontal, up, and down may be used herein with respect to the drawings. However, surgical instruments are used in many orientations and positions, and these terms are not intended to be limiting and/or absolute.

As used herein, the term catheter generally refers to a flexible and thin tubular instrument made of medical grade material designed to be inserted through a narrow opening into an anatomical lumen (e.g., a vessel) to perform a broad range of medical functions. M ore specifically, steerable catheter refers to a medical instrument comprising an elongated shaft made of one or more actuatable sections. As used herein, the term continuum robot and endoscope refer to a steerable rigid or flexible medical instrument which may use light guided by an optical probe to look inside a body cavity or organ.

A medical procedure in which an endoscope is inserted through a natural opening is called endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes. As used herein, the terms optical fiber, fiber optic, or simply fiber refer to an elongated, flexible, light conducting conduit capable of conducting light from one end to another end due to the effect known as total internal reflection. The terms light guiding component or waveguide may also refer to, or may have the functionality of, an optical fiber. The term fiber may refer to one or more light conducting fibers.

Robotic catheters or endoscopes include a flexible tubular shaft operated by an actuating force (pulling or pushing force) applied through drive wires arranged along the tubular shaft and controlled by an actuator unit. The flexible tubular shaft (a/k/a steerable catheter) may include multiple articulated segments configured to continuously bend and turn in a snake-like fashion. Typically, the steerable catheter is inserted through a natural orifice or small incision of a patient's body, and is advanced through a patient's bodily lumen to reach a target site, e.g., a site within the patient's anatomy designated for an intraluminal procedure, such as an ablation or a biopsy. A handheld controller (e.g., a joystick or gamepad controller) may be used as an interface for interaction between the user and the robotic system to control catheter navigation within the patient's body.

Navigation of a steerable catheter can be guided by the live view of a camera or videoscope arranged at the distal tip of the catheter shaft. To that end, a display device, such as an LDC monitor provided in a system console or attached to a wall, displays an image of the camera's FOV image to assist the user in navigating the steerable catheter through the patient's anatomy to reach the target site. The orientation of the camera view, the coordinates of the handheld controller, and the pose or shape of the catheter are mapped (calibrated) before inserting the catheter into the patient's body. As the user manipulates the catheter inside the patient's anatomy, the camera transfers the camera's FOV image to the display device. Ideally, the displayed image should allow the user to relate to the endoscopic image as if the user's own eyes were actually inside the endoscope cavity. Robotic bronchoscopes as described above are increasingly used to screen patients for peripheral pulmonary lesions (PPL) related to lung cancer.

### <Robotic Catheter System>

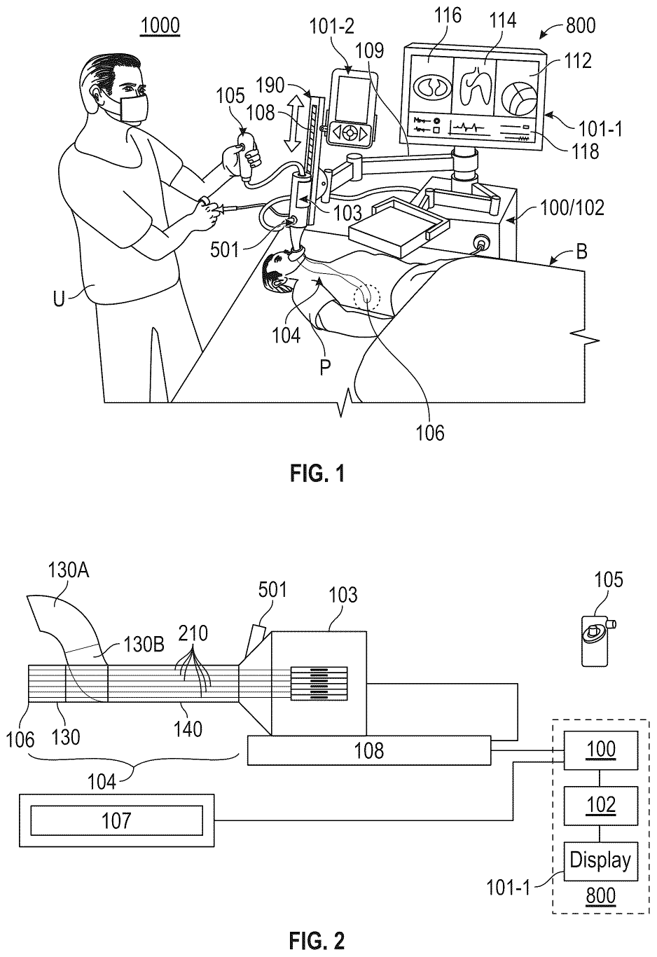

FIG. 1 provides a simplified representation of a medical environment, such as an operating room, where a robotic catheter system 1000 can be used. As illustrated in FIG. 1, the system 1000 may include a system console 800, e.g., computer cart, operatively connected to a steerable catheter 104 via a robotic platform 190. The robotic platform 190 includes one or more than one robotic arm 109 and a linear translation stage 108.

A user U, e.g., a physician, controls the robotic catheter system 1000 via a user interface unit (operation unit) to perform an intraluminal procedure on a patient P positioned on an operating table B. The user interface may include at least one of a main display 101-1 (a first user interface unit), a secondary display 101-2 (a second user interface unit), and a handheld controller 105 (a third user interface unit). The main display 101-1 may include a large display screen attached to the system console 800 or mounted on a wall of the operating room. The secondary display 101-2 may include a compact (portable) display device configured to be removably attached to the robotic platform 190. Examples of the secondary display 101-2 include a portable tablet computer or a mobile communication device (a cellphone).

The steerable catheter 104 is actuated via an actuator unit 103. The actuator unit 103 is attached to the linear translation stage 108 of the robotic platform 190. The handheld controller 105 may include a gamepad controller with a joystick having shift levers and/or push buttons. In an embodiment, the actuator unit 103 may be enclosed in a housing having a shape of a catheter handle. An access port 501 may be provided in or around the catheter handle. The access port 501 may be used for inserting and/or withdrawing end effector tools and/or fluids when performing an interventional procedure of the patient.

The system console 800 includes a system controller 100, a display controller 102, and the main display 101-1. The main display 101-1 may include a conventional display device such as a liquid crystal display (LCD), an OLED display, a QLED display or the like. The main display 101-1 provides a graphic interface unit (GUI) configured to display one or more of a live view image 112, an intraoperative image 114, and a preoperative image 116, and other procedural information 118. The preoperative image 116 may include pre-acquired 3D or 2D medical images of the patient acquired by conventional imaging modalities such as CT, MRI, or ultrasound imaging. The intraoperative image 114 may include images used for image guided procedure such images may be acquired by fluoroscopy or CT imaging modalities.

FIG. 2 is a block diagram of the robotic catheter system 1000.

As illustrated in FIG. 2, a catheter tip position detector 107 is provided in operable communication with the display 100 and the display controller 102. Intraoperative image 114 may be augmented, combined, or correlated with information obtained from the catheter tip position detector 107 and catheter tip tracking sensor 106. The catheter tip tracking sensor 106 may include an electromagnetic (EM) sensor, and the catheter tip position detector 107 may include an EM field generator operatively connected to the system controller 100. Suitable electromagnetic sensors for use with a steerable catheter are well-known and described, for example, in U.S. Pat. No. 6,201,387 and international publication WO 2020/194212 A1.

As illustrated in FIG. 2, the system controller 100 may be operatively connected to the display controller 102, to the actuator unit 103 via the robotic platform 190, and to the tip position detector 107. The tip position detector 107 is at a position spatially related to a three dimensional space where the catheter tip tracking sensor 106 operates. In this manner, the tip position detector 107 is able to track the functional position of the steerable catheter 104.

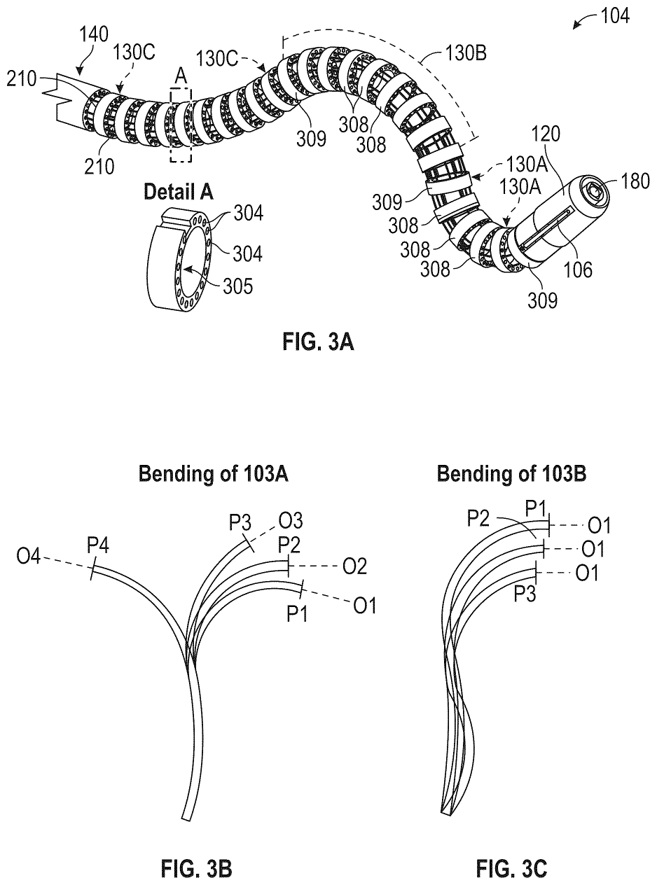

FIG. 3A shows an exemplary embodiment of a steerable catheter 104. The steerable catheter 104 includes a non-steerable proximal section 140, a steerable distal section 130, and a catheter tip 120. The proximal section 140 and distal section 130 are joined to each other by a plurality of drive wires 210 arranged along the wall of the catheter. The proximal section 140 is configured with thru-holes or grooves or conduits to pass drive wires 210 from the distal section 130 to the actuator unit 103. The distal section 130 is comprised of a plurality of bending segments including at least a distal bending segment 130A, a middle bending segment 130B, and a proximal bending segment 130C. Each bending segment is bent by actuation of at least some of the plurality of drive wires 210 (driving members). The posture of the catheter is supported by supporting wires (support members) also arranged along the wall of the catheter. The proximal ends of drive wires 210 are connected to individual actuators or motors of the actuator unit 103, while the distal ends of the drive wires 210 are selectively anchored to anchor members in the different bending segments of the distal section 130.

Each bending segment is formed by a plurality of ring-shaped components (rings) with thru-holes, grooves, or conduits along the wall of the rings. The ring-shaped components may be wire-guiding members 308 or anchor members 309 depending on their function within the catheter. Anchor members 309 are ring-shaped components onto which the distal end of one or more drive wires 210 are attached. Wire-guiding members 308 are ring-shaped components through which some drive wires 210 slide through (without being attached thereto).

Detail “A” in FIG. 3A illustrates an exemplary embodiment of a ring-shaped component (a wire-guiding member 308 or an anchor member 309). Each ring-shaped component includes a central opening which forms a tool channel 305, and plural conduits 304 (grooves, sub-channels, or thru-holes) arranged lengthwise equidistant from the central opening along the annular wall of each ring-shaped component. The non-steerable proximal section 140 is a flexible tubular shaft made of extruded polymer material. The tubular shaft of the proximal section 140 also has a central opening or tool channel and plural conduits along the wall of the shaft surrounding the tool channel 305. In this manner, at least one tool channel 305 formed inside the steerable catheter 104 provides passage for an imaging device and/or end effector tools from the insertion port 501 to the distal end of the steerable catheter 104.

An imaging device 180 that can be inserted through the tool channel 305 includes an endoscope camera (videoscope) along with illumination optics (e.g., optical fibers or LEDs). The illumination optics provides light to irradiate a lesion target which is a region of interest within the patient. End effector tools refer endoscopic surgical tools including clamps, graspers, scissors, staplers, ablation or biopsy needles, and other similar tools, which serve to manipulate body parts (organs or tumorous tissue) during examination or surgery.

The actuator unit 103 may include one or more servo motors or piezoelectric actuators. The actuator unit 103 bends one or more of the bending segments of the catheter by applying a pushing and/or pulling force to the drive wires 210 via a tractor 610. As shown in FIG. 3A, each of the three bendable segments of the steerable catheter 104 has a plurality of drive wires 210. If each bendable segment is actuated by three drive wires 210, the steerable catheter 104 has nine drive wires arranged along the wall of the catheter. Each bendable segment of the catheter is bent by the actuator unit 103 by pushing or pulling at least one of these nine drive wires 210. Force is applied to each individual drive wire in order to manipulate/steer the catheter to a desired pose.

FIG. 3B and FIG. 3C illustrate catheter tip manipulations by actuating one or more bending segments of the steerable catheter 104. As illustrated in FIG. 3B, manipulating only the most distal segment 103A of the steerable section changes the position and orientation of the catheter tip 120. On the other hand, manipulating one or more bending segments (103B or 103C) other than the most distal segment affects only the position of catheter tip 120, but does not affect the orientation of the catheter tip. In FIG. 3B, actuation of distal segment 103A changes the catheter tip from a position P1 having orientation O1, to a position P2 having orientation O2, to position P3 having orientation O3, to position P4 having orientation O4, etc. In FIG. 3C, actuation of the middle segment 103B changes the position of catheter tip 120 from a position P1 having orientation O1 to a position P2 and position P3 having the same orientation O1. Here, it should be appreciated by those skilled in the art that exemplary catheter tip manipulations shown in FIG. 3B and FIG. 3C can be performed during catheter navigation (i.e., while inserting the catheter through tortuous anatomies). The exemplary catheter tip manipulations shown in FIG. 3B and FIG. 3C apply to a targeting mode applied after the catheter tip has been navigated to a predetermined distance (a targeting distance) from the target.

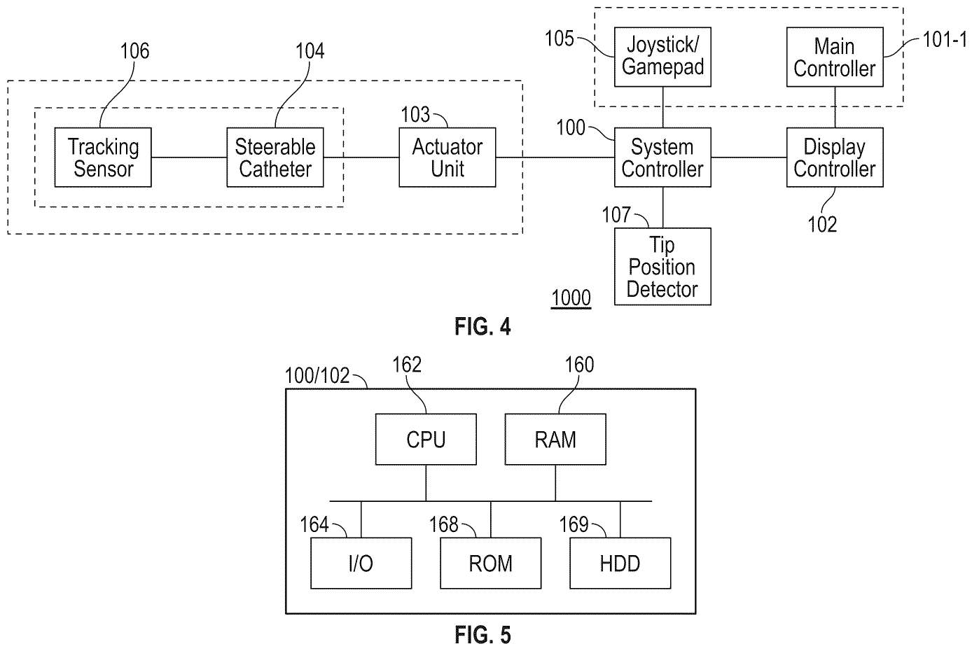

FIG. 4 is a block diagram of the robotic catheter system 1000. As illustrated in FIG. 4, the main display 101-1 and handheld controller 105 are in functional communication with the system controller 100, which is in functional communication with the display controller 102, the actuator unit 103, and the tip position detector 107. The actuator unit 103 is in operable communication with the steerable catheter 104 and the tracking sensor 106.

FIG. 5 is a block diagram of components of the system controller 100 and/or the display controller 102, according to an embodiment. The system controller 100 and the display controller 102 may be configured separately. Alternatively, the system controller 100 and the display controller 102 may be configured as one component. In either case, the system controller 100 and the display controller 102 include substantially the same components. Specifically, the system controller 100 and display controller 102 may include a central processing unit (CPU) 160 comprised of one or more processors (microprocessors), a random access memory (RAM) 162 module, an input/output (I/O) interface 164, a read only memory (ROM) 168, and data storage memory 169, e.g., a hard disk drive (HDD) or solid state drive (SSD).

The ROM 168 and/or HDD 169 may store the operating system (OS) software, and software programs for executing functions of the robotic catheter system 1000 as a whole. The RAM 162 may be used as a workspace memory. The CPU 160 executes the software programs developed in the RAM 162. The I/O 164 inputs, for example, positional information to the display controller 102, and outputs information for displaying the navigation screen to the one or more displays (main display 101-1 and/or secondary display 101-2). The navigation screen may a GUI generated by a software program, may be generated by firmware, or may be a combination thereof.

The system controller 100 may execute software programs and control the display controller 102 to display a navigation screen, e.g., live view image 112. on the main display 101-1 and/or the secondary display 101-2. The display controller 102 may include a graphics processing unit (GPU) or a video display controller (VDC). The display controller 102 may generate a 3D model of an anatomical structure, e.g., a branching structure like an airway of a patient's lungs, based on preoperative or intraoperative images such as CT or MRI images, etc. Alternatively, the 3D model may be received by the system console from another device, e.g., a picture archiving and communication system (PACS) sever. A two dimensional (2D) model may be used instead of 3D model. In this case, the display controller 102 may process, via segmentation, a preoperative 3D image to acquire slice images (2D images) of a patient's anatomy. The 2D or 3D model can be generated before catheter navigation starts. Alternatively, the 2D or 3D model may be generated in real-time (in parallel with the catheter navigation).

The system controller 100 may control the steerable catheter 104 based on any known kinematic algorithms applicable to continuum or snake-like catheter robots. For example, the system controller controls the steerable catheter 104 based on an algorithm known as follow the leader (FTL) algorithm. By applying the FTL algorithm, the most distal segment 130A of the steerable section 130 is actively controlled with forward kinematic values, while the middle segment 130B and the proximal segment 130C (following sections) of the steerable catheter 104 move at a first position in the same way as the distal section moved at the first position or a second position near the first position.

The display controller 102 may acquire position information of the steerable catheter 104 from system controller 100. Alternatively, the display controller 102 may acquire the position information directly from the tip position detector 107. The steerable catheter 104 may be a single-use or limited-use catheter device. In other words, the steerable catheter 104 can be attachable to, and detachable from, the actuator unit 103 to be disposable.

During a procedure, the display controller 102 generates and outputs a live-view image or a navigation screen to the main display 101-1 and/or the secondary display 101-2 based on the 3D model of a patient's anatomy, e.g., a branching structure, and the position information of at least a portion of the catheter, e.g., position of the catheter tip 120, by executing pre-programmed software routines. The navigation screen indicates a current position of at least the catheter tip 120 on the 3D model. By observing the navigation screen, a user can recognize the current position of the steerable catheter 104 in the branching structure. Upon completing navigation to a desired target, one or more end effector tools can be inserted through the access port 501 at the proximal end of the catheter, and such tools can be guided through the tool channel 305 of the catheter body to perform an intraluminal procedure from the distal end of the catheter.

The tool may be a medical tool such as an endoscope camera, forceps, a needle or other biopsy or ablation tools. In an embodiment, the tool may be described as an operation tool or working tool. The working tool may be inserted or removed through the working tool access port 501. The tool may include an endoscope camera or an end effector tool, which can be guided through a steerable catheter under the same principles. A procedure may include a planning procedure, a registration procedure, a targeting procedure, and an operation procedure.

### <Limitations of Motion of Robotic Catheter System>

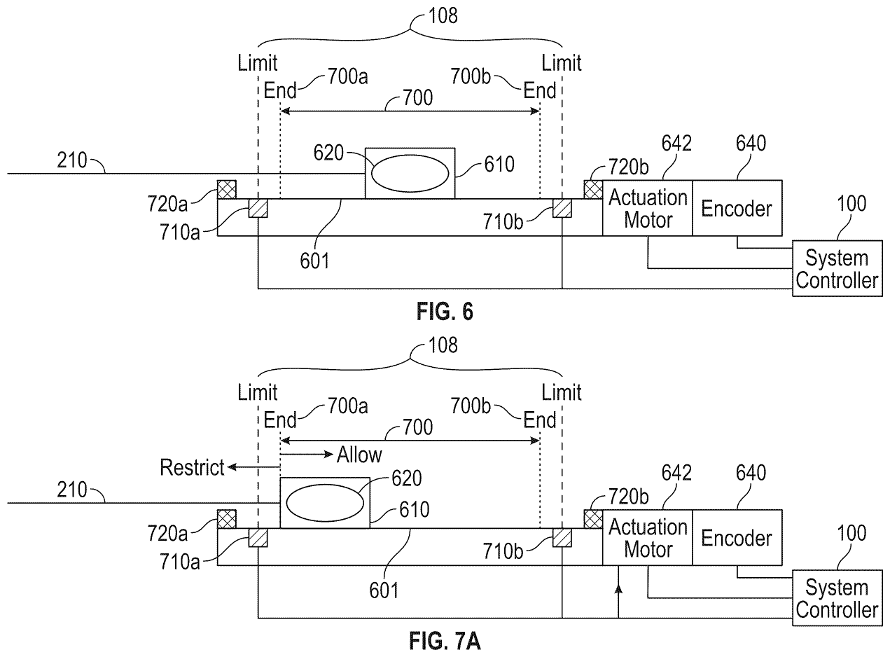

FIG. 6 illustrates movement of a tractor 610 along a track 601, according to an embodiment. As illustrated in FIG. 6, the actuation motor 642 and encoder 640 may extend from an opposite end of the tractor 610. The actuator unit 103 may contain actuation motor 642, encoder 640, track 601, tractor 610, and at least one limit sensor 710a, 710b. At least one wire of the plurality of drive wires 210 extends from one end of the tractor 610, which is configured to at least one of push and pull at least one drive wire of the plurality of drive wires 210.

The tractor 610 may be mounted on the track 601. The track 601 may include a linear bearing, which may convert a rotational motion generated by the actuation motor 642 to a linear push/pull force to propel the tractor 610 along the track 601 within a predetermined range of travel 700. The push/pull force to the drive wire is applied by controlling the position of the tractor 610. The system controller 100 drives the actuation motor 642 and controls the position of the tractor 610 by monitoring signals received from the encoder 640 providing a current position of the tractor 610.

The system controller 100 may control the position of the tractor 610 within the predetermined range of travel 700, which may be set by software limit. As illustrated in FIG. 6, a hard stop 710a, 720b is provided beyond respective ends 700a/700b of the predetermined range of travel 700. Also, a respective one of the at least one limit sensor 710a/710b is provided between the respective end 700a/700b and hard stop 720a/720b.

When the tractor 610 reaches an end 700a, 700b of the predetermined range of travel 700, the system controller 100 restricts movement of the tractor 610 to not move beyond the end 700a/700b of the predetermined range of travel 700. The system controller 100 allows the tractor 610 to move away from the end 700a/700b. Thus, the user U may bend the catheter 104 in any direction that mitigates the bending angle of the bent catheter 104. On the other hand, the user U cannot move further away from the end of the predetermined travel range, with the sensor detecting that the tractor has reached the limit sensor, thus restriction further bending and/or direction of the bend. Thus, the system controller 100 may safely operate, even though the tractor 610 reaches the end 700a/700b of predetermined range of travel 700. The system controller 100 may inform user U when the tractor 610 reaches the end 700a/700b of the predetermined range of travel 700, to assist in navigation/steering of the catheter 104.

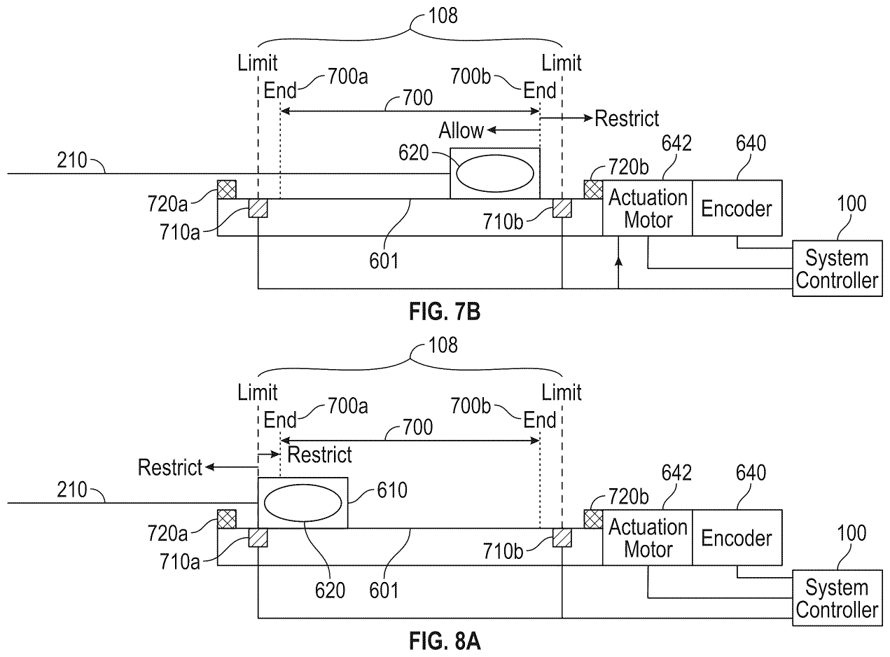

FIG. 7A illustrates movement of the tractor 610 to one end of the predetermined range of travel 700, according to an embodiment. FIG. 7B illustrates movement of the tractor 610 to another end of the predetermined range of travel 700, according to an embodiment.

Limit positions determined by respective limit sensors are located outside of respective ends of the predetermined range of travel 700. As illustrated, the limit positions may be located between the end of travel 700 and the hard stop 720, and may correspond to the position of the limit sensor 710. For conciseness, description of components and operations in FIGS. 7A and 7B that are similar as in FIG. 6 are incorporated here by reference, without repetition of such description.

As illustrated in in FIG. 7A, when the tractor 610 moves past the limit position, outside the predetermined range of travel 700, a limit sensor 720a, 720b sends an alert to the system controller 100, activating a software limit stop that restricts movement of tractor 610 outside the predetermined range of travel 700. The system controller 100 stops linear motion of the tractor 610 until the alert is reset. In an embodiment, the distance from the end of the predetermined range of travel 700 to the position of the limit sensor may be approximately 3 mm to 15 mm. Thus, considering actuation motor 642 response stop time, overshoot of travel distance and resolution of sensor 720a/720b, a limit sensor alert, absent the software stop, may not be provided to the system controller 100 in a timely manner.

The limit sensor alert can be directly sent to a driver of the actuation motor 642 to disable the actuation motor 642, e.g., by cutting the power to the actuation motor 642. In this way, in case the motor is out of control because of the system controller hang, fault or delay, the motor can be disabled with the limit sensors. Thus, for a bending segment having three drive wires that connect to a set of actuation motors 642, the system controller 100 may stop each of the three actuation motors 642 upon receiving an alert generated by the limit sensor 720, and may maintain the stop until reset of the alert.

For a distal section that includes at least a distal bending segment 130A, a middle bending segment 130B, and a proximal bending segment 130C, the system controller 100 may stop all actuation motors 642 associated with each of the bending segments, and may maintain the stop until reset of the alert. Thus, the limit sensor 710 provides a redundant mechanism in case the system controller 100 fails to restrict the tractor 610 upon movement past the end of the predetermined range of travel 700. This redundant limit mechanism ensures safe operation.

A target 620 may be mounted on the tractor or may be configured to move with the tractor, rather than being positioned on the track 601.

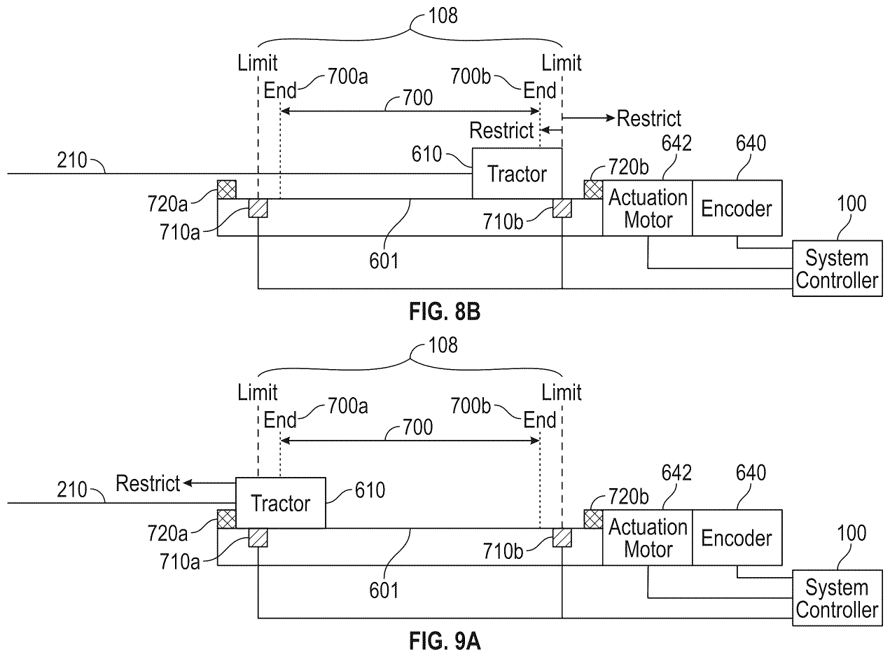

FIG. 8A illustrates preclusion of movement of the tractor outside the predetermined range of travel, according to an embodiment. FIG. 8B illustrates preclusion of movement of the tractor to outside another end of the predetermined range of travel, according to an embodiment.

As illustrated in FIGS. 8A and 8B, an end of the tractor 610 may travel beyond the predetermined range of travel 700, past one of limit sensor 710a or limit sensor 710b. When the tractor 610 reaches a respective limit sensor 710a, 710b, a command is output to stop (restrict) motor movement in either direction. For conciseness, description of components and operations in FIGS. 8A and 8B that are similar as in FIGS. 7A and 7B are incorporated here by reference, without repetition of such description.

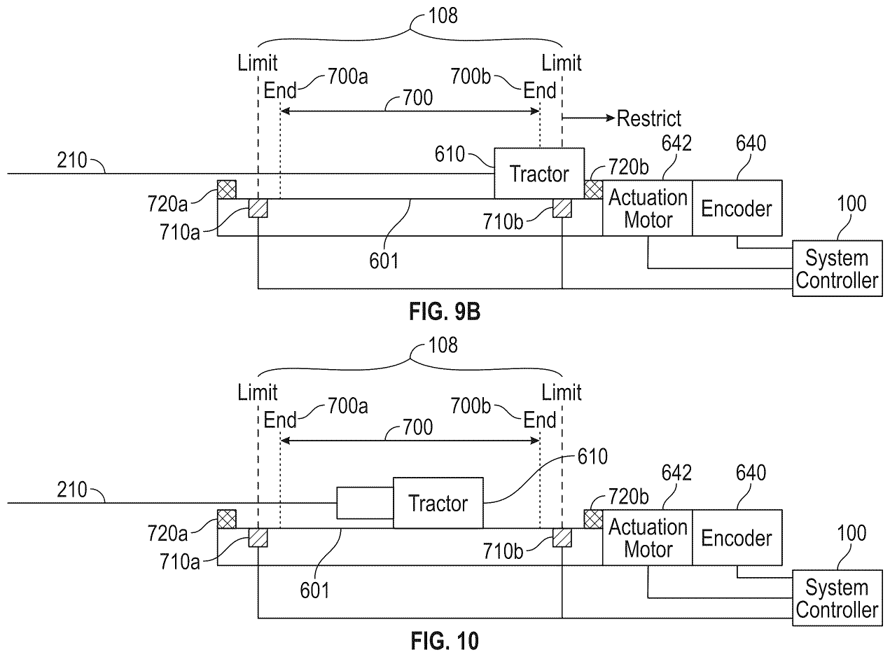

FIG. 9A illustrates preclusion of movement of the tractor beyond a hard stop outside of the predetermined range of travel, according to an embodiment. FIG. 9B illustrates preclusion of movement of the tractor beyond another hard stop outside of the predetermined range of travel, according to an embodiment.

As illustrated in FIGS. 9A and 9B, hard-stops 720a and 720b are positioned outside of the limit positions of the limit sensors 710a, 710b, thus providing a redundant limit mechanism. The hard-stops 720a and 720b mechanically constrain movement of the tractor 610 between hard-stop 720a and hard-stop 720b, without any intervening operation by the system controller 100. In an embodiment, a distance from the position of the limit sensor 710 to the respective hard-stop 720 is within a range of approximately 3 mm and 15 mm.

As illustrated in FIGS. 9A and 9B, the position of the tractor 610 is limited between hard-stops 720a and 720b, thereby ensuring that the push/pull force applied to the drive wire 210 is physically limited, thus providing redundancy for safety operation, in case the actuation motor 642 operations independently or contrary to command from the system controller 100.

For conciseness, description of components and operations in FIGS. 9A and 9B that are similar as in FIGS. 8A and 8B are incorporated here by reference, without repetition of such description.

FIG. 10 illustrates a tractor 610 with a force sensor 740, according to an embodiment. For conciseness, description of components and operations in FIG. 10 that are similar as in FIGS. 9A and 9B are incorporated here by reference, without repetition thereof. As illustrated in FIG. 10, the actuator unit 103, assembled with the steerable catheter 104, is mounted on or adjacent to the linear translation stage 108, which includes a slider and a linear motor. That is, the linear translation stage 108 is motorized and is controlled by the system controller 100 to insert/remove the steerable catheter 104 into/from a patient's bodily lumen. The tractor 610 may include at least one force sensor 740 positioned between the drive wires 210 and the actuation motors 642/actuator 103. The at least one force sensor 740 monitors compression and tensile forces exerted on the drive wires 210. That is, when external forces are applied to the catheter, e.g., by pushing against a patient's organs, force on the drive wires 210 is detected by the force sensor 740. The force sensor 740 may be include strain gauges that convert a mechanical force to electrical resistance. The strain gauges may be arranged as a Wheatstone Bridge circuit to detect changes in electrical resistance with high sensitivity. When applying an excitation voltage to the bridge circuits, changes in electrical resistance are detected as a voltage change. An amplifier may increase the voltage level, i.e., force analog signal, for better signal to noise ratio (SN R) for analog to digital conversion, and the force analog signal is delivered to the system controller 100 after converting to digital signals.

The system controller 100 controls actuation motor 642 for operation of tractor 610 and the drive wires 210, to minimize force analog signals, i.e., force feedback control. To minimize the force analog signals, the plurality of bending segments including at least a distal bending segment 130A, a middle bending segment 130B, and a proximal bending segment 130C of the catheter 104 bend in response to external forces.

The catheter 104 may be robotically controlled in a flexible, relax mode, with the catheter 104 being akin to a soft tube. The user U may change a navigation mode to the relax mode to release external forces on the catheter 104 and/or to minimize forces applied against a patient's organ. The system controller 100 may calculate movements based on input of the force signals and movement commands output to the actuation motor 642. By using the plurality force analog signals with a single common analog to digital converter (ADC), the system controller 100 may use simultaneous force data received from a single bending segment, allowing the system controller 100 to minimize glitches when force data is used for force control feedback.

While in the relax mode, the system controller 100 detects the current position of the tractor 610 by monitoring signals received from the encoder 640. The user U is notified when the system reaches an end of the predetermined range of travel 700, to release external forces thereon. When the tractor 610 reaches a position of a limit sensor 710a, 710b, the system controller 100 may output an alert and transition to an error state. In the error state, the system controller 100 may release a holding torque of the actuation motor 642, to relax the holding force of the drive wire 210. The drive wire 210 may be flexible when control on the actuation motor 642 is released. After eliminating the external forces, the system controller 100 may restart.

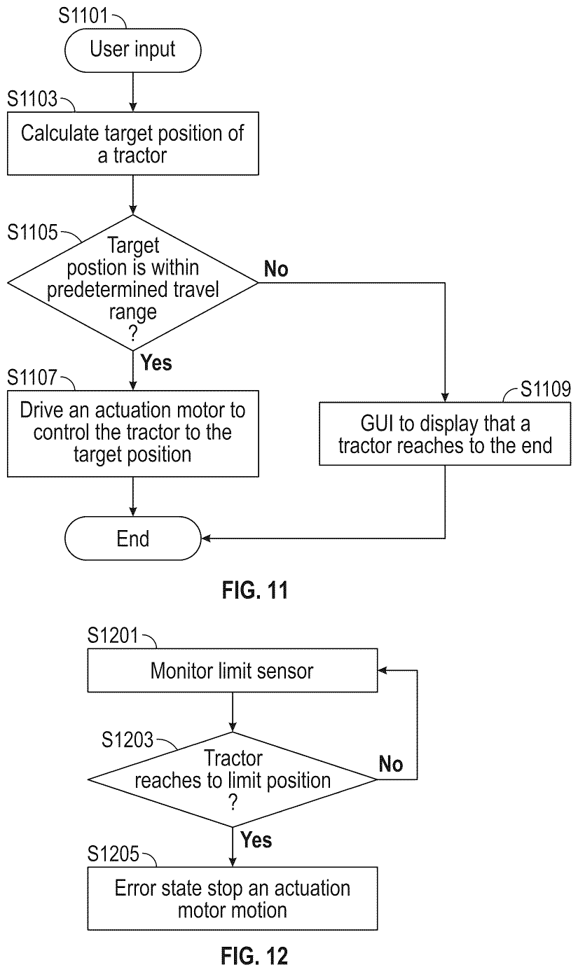

FIG. 11 is a block diagram of a motor drive process according to an embodiment. In Step S1101, a user U may initiate bending of the catheter 104, and in Step S1103 the system controller 100 may calculate a target position of the tractor 610. In Step S1105, the target position may be checked to verify whether the tractor 610 is within the predetermined range of travel 700. If determined to be within the predetermined range of travel 700, in Step S1107 the system controller 100 may control the actuation motor 642 to drive the tractor 610 to the target position. If determined to be out of the predetermined range of travel 700, in Step S1109 the system controller 100 controls output of a notification to the user U via a GUI of the status of the tractor 610, without control the actuation motor 642 to drive the tractor 610, so that the system remains in normal operation even when the tractor 610 reaches the end of the predetermined range of travel 700.

FIG. 12 is a block diagram of operation of a redundant limit mechanism according to an embodiment. As illustrated in FIG. 12, in Step S1201 the system controller 100 monitors the signals received from limit sensors 710a, 710b, regardless of whether the actuation motor 642 is being driven. In Step S1203, the system controller detects a signal from limit sensor 710a/710b indicating that the tractor 610 has reached a limit position and, in Step S1205, the system controller 100 controls output of an alerts and transition into an error state in which the system controller 100 stops all motion of the actuation motor 642. Signals from the limit sensor 710a/710b are independent of signals from the encoder 640, thereby ensuring that the system remains safe regardless of failure of the encoder 640 or a limit sensor 710a/710b.

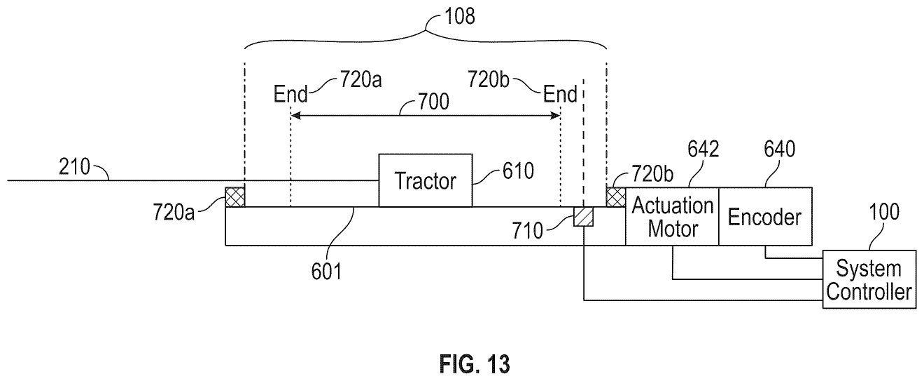

FIG. 13 illustrates components for maintaining the tractor 610 within the predetermined range of travel 700, according to an embodiment. As illustrated in FIG. 2, a tracking sensor 106, e.g., an EM tracking sensor, is attached to the catheter tip 120. Thus, the steerable catheter 104 and the tracking sensor 106 can be tracked by the tip position detector 107. Specifically, the tip position detector 107 detects a position of the tracking sensor 106, and outputs the detected positional information to the system controller 100. The system controller 100 receives the positional information from the tip position detector 107, and continuously records and displays the position of the steerable catheter 104 with respect to the patient's coordinate system. The system controller 100 controls the actuator unit 103 and the linear translation stage 108 in accordance with the manipulation commands input by the user U via one or more of the user interface units (the handheld controller 105, a GUI at the main display 101-1 or touchscreen buttons at the secondary display 101-2).

As illustrated in FIG. 13, only one limit sensor 710 is provided, positioned on one end of the range of travel 700. Providing only one limit sensor 710 simplifies the system and is useful if, e.g., the drive wire 210 requires only a pulling mechanism rather than both a push/pull. Such simplified mechanism requires fewer components and reduces manufacturing cost.

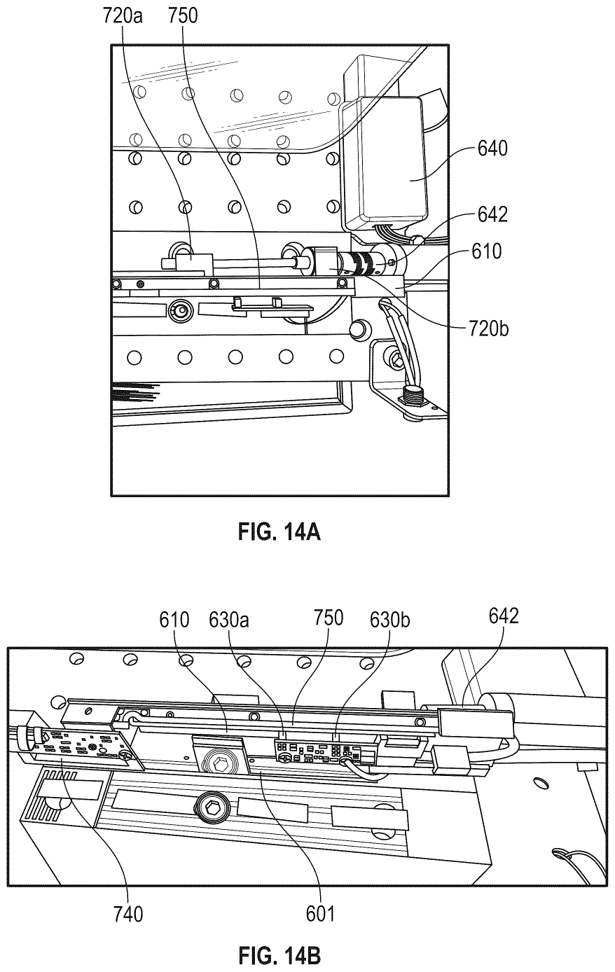

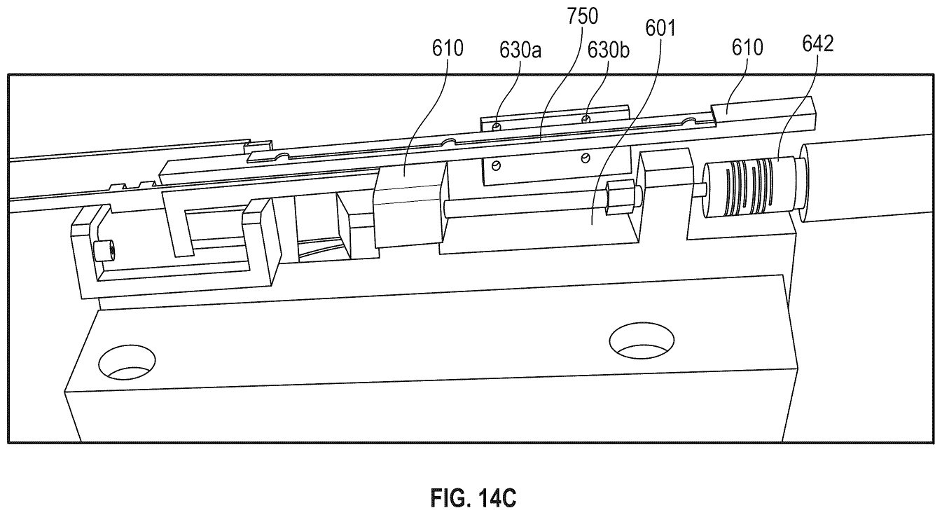

FIG. 14A is a plan view illustrating components of a system according to an embodiment. FIG. 14B is a front perspective view illustrating components of a system according to an embodiment. FIG. 14C is a rear perspective view illustrating components of a system according to an embodiment.

As illustrated in FIGS. 14A-14C, an actuator may include a track 601, a tractor 610, and a sensor 750 that may move with the tractor 610. A first target 630a is provided, positioned adjacent to a first end of the track 601 for detection by the sensor 750. A second target 630b is provided, positioned adjacent to a second end of the track 601 and for detection by the sensor 750. A predetermined range of travel 700 may be set between the first target 630a and the second target 630b.

A motor 642 may also be provided, as well as a first hard stop 720a and a second hard stop 720b, with the first hard stop 720a, the first target 630a, the predetermined range of travel 700, the second target 630b, and the second hard stop 720b being located along the track 601, in such order. Operation of the motor 642 may move the tractor 610 along the track 601 between the first hard stop 720a and the second hard stop 720b. The sensor 750 may detect, as the tractor 610 moves along the track 601, at least one the first target 630a and the second target 630b.

A controller 100 in operational communication with the tractor 610 and the sensor 750 may be provided, as well as an encoder 640 for outputting to the controller 100 data indicating a position of the tractor 610 along the track 601. Data received by the controller 100 from the sensor 750 may be independent of data received by the controller 100 from the encoder 640.

In response to the tractor 610 being within the predetermined range of travel 700, the controller may permit driving of the motor 642, and, in response to the tractor 610 being outside of the predetermined range of travel 700, the controller may control output of a notification of a status of the tractor 610.

An aspect of the present disclosure provides an actuator that includes a track 601, a tractor 610 configured to move along the track 601, a motor 642, a first limit sensor 710a, a second limit sensor 710b, a first hard stop 720a, and a second hard stop 720b. Operation of the motor 642 moves the tractor 610 along the track 601 between the first hard stop 720a and the second hard stop 720b, with the first limit sensor 710a and the second limit sensor 710b being spaced apart along the track 601, between the first hard stop 720a and the second hard stop 720b. The first limit sensor 710a and the second limit sensor 710b may be a switch, a capacitive sensor, a light dependent sensor, a photo sensor, a proximity sensor, and/or a hall effect sensor.

The actuator may also include a controller 100 in operational communication with the motor 642, the first limit sensor 710a, and the second limit sensor 710b. The controller 100 may be configured to receive, from one of the first limit sensor 710a and the second limit sensor 710b, data corresponding to a position of the tractor 610 along the track 601; detect, based on the received data, the tractor 610 being located at one of a first position or a second position. The first position is set by software limit. The second position may be at or adjacent to one of the first limit sensor 710a or the second limit sensor 710b; and a third position is at at least one hard stop, which may be adjacent to the first limit sensor 710a.

In response to detection of the tractor 610 at the second position, the motor 642 is controlled to move the tractor 610 away from the third position. In response to detection of the tractor 610 at the second third, the motor 642 is controlled to move bring the tractor 610 to a hard stop.

A predetermined range of travel 700 may be set between the first limit sensor 710a and the second limit sensor 710b. In response to the tractor 610 being within the predetermined range of travel 700, the controller may be configured to permit driving of the motor 642, and, in response to the tractor 610 being outside of the predetermined range of travel 700, the controller may be configured to control output of a notification of a status of the tractor 610.

In response to the notification, a user U may disable the motor to stop movement of the tractor 610.

A target 620 may be provided that is configured to move with the tractor 610, with at least one of the first limit sensor 710a and the second limit sensor 710b being configured to detect the target 620 as the tractor 610 moves along the track 601 past a respective at least one of the first limit sensor 710a and the second limit sensor 710b.

The target 620 may be provided on a predetermined position on a side of the tractor 610, and the first limit sensor 710a and the second limit sensor 710b may be located on a side of the track 601 facing the side of the tractor on which the target 620 is provided. The first hard stop 720a and the second hard stop 720b may be located on a side of the track 601 opposite to the side of the track 601 facing the side of the tractor on which the target 620 is embedded.

An aspect of the present disclosure provides an actuator that includes a track 601; at least one limit sensor 710a, 710b; at least one hard stop 720a, 720b; a tractor 610 moveable along the track 601 towards or away from the at least one hard stop 720a, 720b; a controller 100 in operational communication with the at least one limit sensor 710a, 710b; and a target 620 at least one of positioned on the tractor 610 and configured to move with the tractor 610. The at least one limit sensor 710a, 710b may be configured to detect at least one of approach and retreat of the target 620 as the tractor moves along the track 601. The controller may be configured to: receive, from the at least one limit sensor, data corresponding to a position of the tractor; detect, based on the received data, the tractor being located adjacent to the at least one limit sensor; and, in response to detecting the tractor at the adjacent location, control to move the tractor away from each of the at least one limit sensor and the at least one hard stop.

A Iso provided is a surgical apparatus that includes the above disclosed actuator; and a continuum robot including a catheter body having a proximal portion 140, an articulated distal portion 130, and a plurality of drive wires 210. The tractor 610 may be configured to attach to a proximal end of a drive wire of the plurality of drive wires 210, and movement of the tractor 610 alters a pose of the articulated distal portion 130.

A further aspect of the disclosure provides an actuator that includes a track 601, a tractor 610, a sensor 750 configured to move with the tractor 610, a first target 630a positioned adjacent to a first end of the track 601 and configured to be detected by the sensor 750, and a second target 630b positioned adjacent to a second end of the track 601 and configured to be detected by the sensor 750, with a predetermined range of travel 700 being set between the first target 630a and the second target 630b.

The actuator may also include a motor 642, a first hard stop 720a, and a second hard stop 720b, with the first hard stop 720a, the first target 630a, the predetermined range of travel 700, the second target 630b, and the second hard stop 720b being located along the track 601, in such order. Operation of the motor 642 may move the tractor 610 along the track 601 between the first hard stop 720a and the second hard stop 720b, and the sensor 750 may be configured to detect, as the tractor 610 moves along the track 601, at least one the first target 630a and the second target 630b.

The actuator may also include a controller 100 in operational communication with the tractor 610 and the sensor 750; and an encoder 640 configured to output to the controller 100 data indicating a position of the tractor 610 along the track 601. Data received by the controller 100 from the sensor 750 may be independent of data received by the controller 100 from the encoder 640. In response to the tractor 610 being within the predetermined range of travel 700, the controller may permit driving of the motor 642, and, in response to the tractor 610 being outside of the predetermined range of travel 700, the controller may control output of a notification of a status of the tractor 610.

A still further aspect of the present disclosure provides a surgical apparatus that includes a continuum robot having a catheter body having a proximal portion 140, an articulated distal portion 130, and a plurality of drive wires 210; a plurality of tractors, with each tractor 610 of the plurality of tractors configured to change poses of the articulated distal portion 130 by operation of a respective at least one drive wire of the plurality of drive wires 210; a plurality of motors, each motor 642 of the plurality of motors being operatively coupled with a respective tractor 610; a linear transition stage 108 including a track 601; a first limit sensor 710a configured to detect at least one of approach and retreat of the tractor 610 to/from a first position along the linear transition stage 108; a second limit sensor 710b configured to detect at least one of approach and retreat of the tractor 610 to/from a second position along the linear transition stage 108; and a controller 100 in operational communication with each motor 642 of the plurality of motors, each tractor 610 of the plurality of tractors, the first limit sensor 710a, and the second limit sensor 710b.

The controller 100 may be configured to receive, from one of the first limit sensor 710a and the second limit sensor 710b, data corresponding to a position of the tractor 610 along the linear transition stage 108; and determine, based on the received data, the tractor 610 being located at one of a first position adjacent to the first limit sensor 710a and a second position adjacent to the second limit sensor 710b. In response to the tractor 610 determined to be at the first position, the controller may control the motor 642 to move the tractor 610 towards the second position; and, in response to the tractor 610 determined to be at the second position, the controller may control the motor 642 to move the tractor 610 towards the first position.

A signal for controlling the motor 642 to one of move the tractor 610 towards the second position and move the tractor 610 towards the first position may be input directly to the motor 642 from one of the first limit sensor 710a and the second limit sensor 710b.

A predetermined range 700 may be provided between the first position and the second position. In response to the received data indicating that at least a part of the tractor 610 is located outside of the predetermined range 700 and beyond one of the first limit sensor 710a and the second limit sensor 710b, the controller 100 may control the motor 642 to move the tractor 610 towards the second position and the first position, respectively.

The controller may be configured to stop operation of each motor 642 of the plurality of motors in response to (a) the received data indicating that at least a part of the tractor 610 is located beyond the first position adjacent on a side of the first limit sensor 710a opposite to the second limit sensor 710b, or (b) the received data indicating that at least a part of the tractor 610 is located beyond the second position adjacent on a side of the second limit sensor 710b opposite to the first limit sensor 710a.

Thus, a robotic control system is provided that includes a system controller 100 which controls driving of the tractor 610, with one or more controllers that control the tractor 610 to move within a predetermined range of travel 700. A sensor is provided that is configured to detect when the tractor 610 reaches a limit position, with the limit position being outside of the predetermined range of travel 700. When the tractor 610 reaches an end of the predetermined range of travel 700, the system controller 100 restricts the tractor 610 when moving over the end of the range of travel 700 while the controllers allow the tractor 610 to move away from the end of the predetermined range of travel 700. When a sensor detects that the tractor 610 reaches to a limit position, the system controller 100 restricts movement the tractor 610 past the limit while allowing the tractor 610 to move away from the limit. Thus, the system controller 100 ensure safe and reliable operated with redundant limit mechanisms.

Thus, an aspect of the present disclosure provides an actuator that includes a track; a tractor; a motor; an encoder; at least one limit sensor; and a controller in operational communication with the motor, the encoder, and the at least one limit sensor. Operation of the motor moves the tractor along the track at least between a first position, a second position and a third position. The first position is set on a first side of the least one limit sensor. The second position spaced apart from the first position. The third position is set on a second side of the least one limit sensor that is opposite the first side. The encoder is configured to output data indicating a position of the tractor at at least at one of the first position, the second position and the third position.

The first position may be a software limit, the second position may be at or adjacent to the at least one limit sensor, and the third position may be at least one hard stop adjacent to the first hardware limit. A range of travel of the tractor may be defined by the software limit. In response to the tractor being within the range of travel, the controller permits driving of the motor. In response to the tractor being outside of the range of travel, the controller control output of a notification of an error state. The at least one hard stop may physically restrict movement of the tractor beyond an end of the range of travel opposite the first position.

The controller may receive the data indicating the position of the tractor along the track and detect, based on the received data, a location of the tractor. In response to detecting the tractor at the third position, the controller may permit the motor to move the tractor towards the second position. In response to detecting the tractor at the second position, the controller may restrict the motor from moving the tractor towards the third position. In response to detecting the tractor at the third position, the controller may enter an error state and stop the motor. The at least one limit sensor may be at least one of a switch, a capacitive sensor, a light dependent sensor, a photo sensor, a proximity sensor, and a hall effect sensor.

The actuator may include a target configured to move with the tractor, with the at least one limit sensor being configured to detect the target. The controller may receive the data based on proximity of the target to the at least one limit sensor, and the data received by the controller from the at least one limit sensor is independent of the data output by the encoder. The target may be provided on a predetermined position on a side of the tractor, and the at least one limit sensor may be located on a side of the track facing the side of the tractor on which the target is provided. At least one hard stop may be located on a side of the track opposite to the side of the track facing the side of the tractor on which the target is embedded.

A surgical apparatus may be provided that includes a continuum robot including a catheter body having a proximal portion, an articulated distal portion, and at least one drive wire; and the above described actuator, with the tractor being configured to attach to a proximal end of the at least one drive wire. movement of the tractor alters a posture and/or pose of the articulated distal portion.

An aspect of the present disclosure provides a method for controlling an actuator that includes a track, a tractor, a motor, an encoder, and at least one limit sensor. The method includes operating the motor to move the tractor along the track at least between a first position, a second position and a third position, and outputting, by the encoder, data indicating a position of the tractor at at least at one of the first position, the second position and the third position. The first position may be set on a first side of the least one limit sensor. The second position may be spaced apart from the first position. The third position may be set on a second side of the least one limit sensor that is opposite the first side. In response to detecting the tractor at the third position, the controller enters an error state and stops the motor from moving the tractor. The method also includes setting a software limit to define a range of travel of the tractor, with the first position being a software limit, the second position being at or adjacent to the at least one limit sensor, and the third position being at least one hard stop. In response to the tractor being within the range of travel, driving of the motor is permitted, and in response to the tractor being outside of the range of travel, a notification of an error state is output.

A further aspect of the present disclosure provides an actuator that includes a plurality of tracks; a plurality of physical stops at one end of each track of the plurality of tracks; a plurality of tractors, with each tractor of the plurality of tractors configured to travel along a respective track of the plurality of tracks; a plurality of motors, with each motor of the plurality of motors configured to drive a respective tractor; a plurality of encoders, with each encoder of the plurality of encoders being configured to encode a position of a respective tractor along the respective track; a plurality of limit sensors; and a controller in operational communication with the plurality of motors, the plurality of encoders, and the plurality of limit sensors. Operation of a first motor moves a first tractor along a first track at least between a first position, a second position and a third position, provided in such order along the first track. A first encoder of the plurality of encoders may be configured to output data indicating a position of the first tractor at least between the first position and the second position. The first position may be set as a software limit. The second position may be set at a first predetermined position at or adjacent to a first limit sensor along the first track. The third position may be set at a second predetermined position at or adjacent to a first physical stop along the track. A range of travel of the first tractor may be based on the set software limit. In response to the first tractor being within the range of travel, the controller may permit driving of the motor. In response to the first tractor being outside of the range of travel, the controller may control output of an error state notification. In response to detecting the first tractor at the second position, the controller may restrict the first motor from moving the first tractor towards the third position.

The actuator may also include a second track of the plurality of tracks; a second physical stop of the plurality of physical stops; a second tractor of the plurality of tractors; a second motor of the plurality of motors; a second encoder of the plurality of encoders; and a second limit sensor of the plurality of limit sensors. Operation of the second motor moves the second tractor along the second track at least between the first position, the second position and the third position. The second encoder may output data indicating a position of the second tractor at least between the first position and the second position. In response to detecting the first tractor or the second at the third position, the controller may stop travel of the first tractor and stop travel of the second tractor.

In response to the first tractor being outside of the range of travel, the controller may release a holding torque of each motor of the plurality of motors. The plurality of motors may control one of a posture and/or pose of a same articulated portion of a continuum robot driven by the actuator. The release of the holding torque relaxes a holding force of each drive wire driven by each respective tractor of the plurality of tractors.

### REFERENCE NUMBERS

| | |
|---|---|
| 100 | system controller |
| 101-1 | main display / first user interface unit |
| 101-2 | secondary display / second user interface unit |
| 102 | display controller |
| 103 | actuator unit |
| 104 | steerable catheter / continuum robot |
| 105 | handheld controller / third user interface unit |
| 106 | catheter tip tracking sensor |
| 107 | catheter tip position detector |
| 108 | linear translation stage |
| 109 | robotic arm |
| 112 | view image |
| 114 | intraoperative image |
| 116 | preoperative image |
| 118 | procedural information |
| 120 | catheter tip |
| 130 | steerable distal section |
| 140 | non-steerable proximal section |
| 160 | CPU |
| 162 | RAM |
| 164 | I/O interface |
| 168 | ROM |
| 169 | HDD/SSD |
| 180 | imaging device |
| 190 | robotic platform |
| 210 | plurality of drive wires |
| 304 | plural conduits |
| 305 | tool channel |
| 308 | wire-guiding members |
| 309 | anchor members |
| 501 | access port |
| 601 | track |
| 610 | tractor |
| 620 | target |
| 630a, 630b | targets |
| 640 | encoder |
| 642 | actuation motor |
| 700 | range of travel |
| 710a, 710b | limit sensors |
| 720a, 720b | hard-stops |
| 740 | force sensor |
| 750 | tractor position sensor |
| 800 | system console |
| 1000 | robotic catheter system |
| U | user |
| P | patient |
| B | operating table |

The terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

Embodiments can be realized by a computer of a system or apparatus that reads out and executes computer executable instructions (e.g., one or more programs) recorded on a storage medium (which may also be referred to more fully as a non-transitory computer-readable storage medium) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., a CPU, micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a RAM, a ROM, a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The detector interface also provides communication interfaces to input and output devices. The detector may include, for example a photomultiplier tube (PMT), a photodiode, an avalanche photodiode detector (APD), a charge-coupled device (CCD), multi-pixel photon counters (MPPC), or the like. Also, the function of detector may be realized by computer executable instructions (e.g., one or more programs) recorded on a storage/RAM.

In referring to the description, specific details are set forth in order to provide a thorough understanding of the examples disclosed. In other instances, well-known methods, procedures, components, and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

### EXEMPLARY REFERENCES

The present disclosure provide significant useful improvements of conventional systems and methods of catheter guidance. Readers of the disclosure may review the following patent and non-patent publications. U.S. Pat. Nos. 2,975,785, 3,253,524, 3,610,231, 3,788,303, 4,207,873, 5,355,871, 9,737,373, 10,470,831, US20100041949, US20120078053, US20190261830, Pierre Berthet-Rayne, et al., “The i2Snake Robotic Platform for Endoscopic Surgery”, Ann Biomed Eng. 2018 October; 46(10):1663-1675, 2018; and Esther D. Rozeboom, et al., “Feasibility of joystick guided colonoscopy”, J Robotic Surg. 9:173-178, 2015.

Each of the foregoing patent and non-patent publications is incorporated by reference herein in their entirety.

What is claimed is:

1. An actuator comprising:
a track;
a tractor;
a motor;
an encoder;
at least one limit sensor; and
a controller in operational communication with the motor, the encoder, and the at least one limit sensor, wherein:
operation of the motor moves the tractor along the track at least between a first position, a second position and a third position,
the first position is set on a first side of the least one limit sensor,
the second position spaced apart from the first position,
the third position is set on a second side of the least one limit sensor that is opposite the first side,
the encoder is configured to output data indicating a position of the tractor at at least at one of the first position, the second position and the third position.

2. The actuator of claim 1, wherein:
the first position is a software limit,
the second position is at or adjacent to the at least one limit sensor, and
the third position is at least one hard stop adjacent to the first hardware limit.

3. The actuator of claim 2, wherein a range of travel of the tractor is defined by the software limit.

4. The actuator of claim 3, wherein:
in response to the tractor being within the range of travel, the controller is configured to permit driving of the motor, and
in response to the tractor being outside of the range of travel, the controller is configured to control output of a notification of an error state.

5. The actuator of claim 3, wherein the at least one hard stop is configured to physically restrict movement of the tractor beyond an end of the range of travel opposite the first position.

6. The actuator of claim 1, wherein the controller is configured to:
receive the data indicating the position of the tractor along the track; and
detect, based on the received data, a location of the tractor.

7. The actuator of claim 6, wherein, in response to detecting the tractor at the third position, the controller is configured to permit the motor to move the tractor towards the second position.

8. The actuator of claim 6, wherein, in response to detecting the tractor at the second position, the controller is configured to restrict the motor from moving the tractor towards the third position.

9. The actuator of claim 6, wherein, in response to detecting the tractor at the third position, the controller is configured to enter an error state and stop the motor.

10. The actuator of claim 1, wherein the at least one limit sensor is at least one of a switch, a capacitive sensor, a light dependent sensor, a photo sensor, a proximity sensor, and a hall effect sensor.

11. The actuator of claim 1, further comprising a target configured to move with the tractor,
wherein the at least one limit sensor is configured to detect the target.

12. The actuator of claim 11, wherein:
the controller is configured to receive the data based on proximity of the target to the at least one limit sensor, and
the data received by the controller from the at least one limit sensor is independent of the data output by the encoder.

13. The actuator of claim 11, wherein:
the target is provided on a predetermined position on a side of the tractor, and
the at least one limit sensor is located on a side of the track facing the side of the tractor on which the target is provided.

14. The actuator of claim 11, wherein at least one hard stop is located on a side of the track opposite to the side of the track facing the side of the tractor on which the target is embedded.

15. A surgical apparatus comprising:
a continuum robot including a catheter body having a proximal portion, an articulated distal portion, and at least one drive wire; and
the actuator of claim 1, wherein:
the tractor is configured to attach to a proximal end of the at least one drive wire, and
movement of the tractor alters at least one of a posture and/or pose of the articulated distal portion.

16. A method for controlling an actuator that includes a track, a tractor, a motor, an encoder, and at least one limit sensor, the method comprising:
operating the motor to move the tractor along the track at least between a first position, a second position and a third position; and
outputting, by the encoder, data indicating a position of the tractor at at least at one of the first position, the second position and the third position, wherein:
the first position is set on a first side of the least one limit sensor,
the second position is spaced apart from the first position, and
the third position is set on a second side of the least one limit sensor that is opposite the first side.

17. The method of claim 16, further comprising:
in response to detecting the tractor at the third position, entering an error state and stopping the motor from moving the tractor.

18. The method of claim 16, further comprising:
setting a software limit to define a range of travel of the tractor, with the first position being a software limit, the second position being at or adjacent to the at least one limit sensor, and the third position being at least one hard stop.

19. The method of claim 18, wherein:
in response to the tractor being within the range of travel, driving of the motor is permitted, and
in response to the tractor being outside of the range of travel, a notification of an error state is output.

20. An actuator comprising:
a plurality of tracks;
a plurality of physical stops at one end of each track of the plurality of tracks;
a plurality of tractors, with each tractor of the plurality of tractors configured to travel along a respective track of the plurality of tracks;
a plurality of motors, with each motor of the plurality of motors configured to drive a respective tractor;
a plurality of encoders, with each encoder of the plurality of encoders being configured to encode a position of a respective tractor along the respective track;
a plurality of limit sensors; and
a controller in operational communication with the plurality of motors, the plurality of encoders, and the plurality of limit sensors, wherein:
operation of a first motor moves a first tractor along a first track at least between a first position, a second position and a third position, provided in such order along the first track,
a first encoder of the plurality of encoders is configured to output data indicating a position of the first tractor at least between the first position and the second position,
the first position is set as a software limit,
the second position is set at a first predetermined position at or adjacent to a first limit sensor along the first track,
the third position is set at a second predetermined position at or adjacent to a first physical stop along the track,
a range of travel of the first tractor is based on the set software limit,
in response to the first tractor being within the range of travel, the controller is configured to permit driving of the motor,
in response to the first tractor being outside of the range of travel, the controller is configured to control output of an error state notification, and
in response to detecting the first tractor at the second position, the controller restricts the first motor from moving the first tractor towards the third position.

21. The actuator of claim 20, further comprising:
a second track of the plurality of tracks;
a second physical stop of the plurality of physical stops;
a second tractor of the plurality of tractors;
a second motor of the plurality of motors;
a second encoder of the plurality of encoders; and
a second limit sensor of the plurality of limit sensors, wherein:
operation of the second motor moves the second tractor along the second track at least between the first position, the second position and the third position,
the second encoder is configured to output data indicating a position of the second tractor at least between the first position and the second position, and
in response to detecting the first tractor or the second at the third position, the controller stops travel of the first tractor and stops travel of the second tractor.

22. The actuator of claim 20, wherein, in response to the first tractor being outside of the range of travel, the controller is configured to release a holding torque of each motor of the plurality of motors.

23. The actuator of claim 22, wherein the plurality of motors are configured to control one of a posture and/or pose of a same articulated portion of a continuum robot driven by the actuator.

24. The actuator of claim 22, wherein the release of the holding torque relaxes a holding force of each drive wire driven by each respective tractor of the plurality of tractors.
